(19) United States
(12) Patent Application Publication
(10) Pub. No.: US 2011/0275577 A1
(43) Pub. Date: Nov. 10, 2011

(54) METHODS OF TREATING DERMATOLOGIC, GYNECOLOGIC, AND GENITAL DISORDERS WITH CAFFEIC ACID ANALOGS

(75) Inventors: Anna Priebe, Sioux Falls, SD (US); Waldemar Priebe, Houston, TX (US)

(73) Assignee: Moleculin, LLC, Houston, TX (US)

(21) Appl. No.: 12/987,553

(22) Filed: Jan. 10, 2011

Related U.S. Application Data
(60) Provisional application No. 61/293,605, filed on Jan. 8, 2010.

(51) Int. Cl. A61K 31/44; A61K 31/4402; A61K 31/4406; A61K 31/216; A61K 31/165; A61P 15/00; A61K 31/7028; A61K 31/56; A61K 31/59; A61P 17/00; A61P 15/02; A61P 35/00; A61K 31/277; A61K 31/4164

(52) U.S. Cl. 514/25; 514/357; 514/521; 514/532; 514/622; 514/400; 514/171; 514/167

(57) ABSTRACT

The present invention relates to caffeic acid analog compounds and methods which may be useful for regeneration, cellular programming, and the treatment of dermatologic, gynecologic, and genital diseases such as inflammatory dermatologic conditions, dysplasia, neoplasia, in situ carcinoma, invasive carcinoma, lichen sclerosus, lichen planus, vaginal dysplasia, vaginal carcinoma, vulvar dysplasia, vulvar carcinoma, cervical dysplasia, cervical carcinoma, and Kaposi's sarcoma.

This application claims the benefit of priority of U.S. provisional application No. 61/293,605, filed Jan. 8, 2010, the disclosure of which is hereby incorporated by reference as if written herein in its entirety.

### FIELD OF INVENTION

Disclosed herein are new caffeic acid analog compounds and compositions and their application as pharmaceuticals for the treatment of disease. Methods of inhibition of STAT activity in a human or animal subject are also provided for the treatment of dermatologic, gynecologic, and genital diseases such as inflammatory dermatologic conditions, dysplasia, neoplasia, in situ carcinoma, invasive carcinoma, lichen sclerosus, lichen planus, vaginal dysplasia, vaginal carcinoma, vulvar dysplasia, vulvar carcinoma, cervical dysplasia, cervical carcinoma, and Kaposi's sarcoma.

### BACKGROUND OF THE INVENTION

STAT's (STAT3 and STATS particularly) are up regulated in many cancers including glioblastoma, head and neck cancer head, prostate cancer, leukemias and breast cancer. A constitutively active form of STAT3 is oncogenic, though these mutations have not been identified in human cancer as yet. STAT3 activation is associated with a number of inflammatory diseases of the skin, gut, respiratory system and brain; such as psoriasis, Crohn's disease, inflammatory bowel disease (IBD), pulmonary fibrosis and acute lung injury, as well as multiple sclerosis (M.S.). STAT3 is also critical for leptin signaling and its mutation leads to obesity in mice.

Under normal physiological conditions, latent STAT3 activation is dependent on ligand-receptor interaction, primarily under the control of growth factor receptor tyrosine kinases or cytokine and G-protein receptors with associated Jak2. Winston L A, Hunter T, JAK2, Ras, and Raf Are Required For Activation Of Extracellular Signal-Regulated Kinase/Mitogen-Activated Protein Kinase By Growth Hormone, J Biol Chem 1995; 270:30837-30840.

In the majority of CNS melanoma metastases and primary brain tumors, for example, STAT3 is constitutively active. As noted above, the STAT3 pathway, however, can also be induced by cytokines such as IL-6, which is expressed in the CNS under a variety of conditions and by a variety of growth factors. Activation of the STAT3 pathways results in nuclear translocation and subsequent translation of key factors that are responsible for proliferation, resistance to apoptosis, and invasion/metastasis.

Specifically, the epidermal growth factor receptor (EGFR), interleukin (IL)-6, or IL-4 activate STAT3 by phosphorylation of the tyrosine residue in the transactivation domain of STAT3. Mizoguchi M, Betensky R A, Batchelor T T, Bernay D C, Louis D N, Nutt C L, Activation of STAT3, MAPK, and AKT in Malignant Astrocytic Gliomas: Correlation with EGFR Status, Tumor Grade, And Survival, J Neuropathol Exp Neurol 2006; 65:1181-1188; Rahaman S O, Harbor P C, Chemova O, Barnett G H, Vogelbaum M A, Haque S J, Inhibition Of Constitutively Active Stat3 Suppresses Proliferation And Induces Apoptosis In Glioblastoma Multiforme Cells, Oncogene 2002; 21:8404-8413.

Other non-receptor tyrosine kinases, such as v-src and v-abl, can also activate STAT3 and are among the most frequently activated oncogenic proteins. Upon tyrosine phosphorylation (p-STAT3), dimers of STAT3 are formed, translocate into the nucleus, and induce the expression of a variety of transcriptional factors. Whereas tyrosine phosphorylation of STAT3 regulates dimerization, nuclear translocation, and DNA binding, serine/threonine phosphorylation optimizes transcriptional activity. Turkson J, Ryan D, Kim J, Zhang Y, Chen Z, Haura E, Laudano A, Sebti S, Hamilton A, Jove R, Phosphotyrosyl Peptides Block Stat3-Mediated DNA Binding Activity, Gene Regulation, And Cell Transformation, J Biol Chem 2001; 276:45443-45455.

STAT3, promotes tumorigenesis by preventing apoptosis (by increasing survivin, BCL-XL, and MCL1 expression) and enhancing proliferation (by increasing c-Myc and cyclin D1/D2 expression), angiogenesis (by increasing VEGF and HIF-1α expression), invasion (by increasing MMP-2 and MMP-9 expression), and metastasis and is a key regulator of immunosuppression. Masamune A, Satoh M, Kikuta K, Suzuki N, Shimosegawa T, Activation of JAK-STAT Pathway Is Required For Platelet-Derived Growth Factor-Induced Proliferation Of Pancreatic Stellate Cells, World J Gastroenterol 2005; 11:3385-3391; Yu H, Jove R, The STATs Of Cancer—New Molecular Targets Come Of Age, Nat Rev Cancer 2004; 4:97-105; Huang S, Regulation Of Metastases By Signal Transducer And Activator Of Transcription 3 Signaling Pathway: Clinical Implications, Clin Cancer Res 2007; 13:1362-1366; Yu H, Kortylewski M, Pardoll D, Crosstalk Between Cancer And Immune Cells: Role Of STAT3 In The Tumour Microenvironment, Nat Rev Immunol 2007; 7:41-51.

Furthermore, in regulatory T cells (Tregs), the cytokine IL-2 has been shown to activate STAT3, resulting in transcriptional activation of FoxP3, which has been correlated with functional immunosuppressive activity. The activation of STAT3 has also been shown to induce the immunosuppressive cytokine transforming growth factor (TGF)-β and inhibit dendritic cell maturation, the expression of co-stimulatory molecules, and effector T cell proliferation responses. Therefore, blockade of activation of STAT3 and its subsequent nuclear translocation inhibits both tumorigenesis and tumor-mediated immunosuppression.

Investigators have examined activated STAT3 (herein sometimes referred to as “p-STAT3”) expression in malignancies such as gastric, renal, and ovarian cancers; squamous cell and hepatocellular carcinoma; and anaplastic large cell lymphoma and have determined that p-STAT3 phosphorylation at tyrosine 705 correlates with poor prognosis. Other studies have shown that the expression of p-STAT3 correlates with lymph node spread and depth of invasion in colorectal cancer. In contrast, some studies of non-small cell lung cancer and gliomas have shown no relationship between p-STAT3 expression and prognosis. Mizoguchi M, Betensky R A, Batchelor T T, Bernay D C, Louis D N, Nutt C L, Activation Of STAT3, MAPK, And AKT In Malignant Astrocytic Gliomas: Correlation With EGFR Status, Tumor Grade, And Survival, J Neuropathol Exp Neurol 2006; 65:1181-1188; Gong W, Wang L, Yao J, Ajani J A, Wei D, Aldape K, Xie K, Sawaya R, Huang S, Expression Of Activated Signal Transducer And Activator Of Transcription 3 Predicts Expression Of Vascular Endothelial Growth Factor In And Angiogenic Phenotype Of Human Gastric Cancer, Clin Cancer Res 2005; 11:1386-1393; Horiguchi A, Oya M, Shimada T, Uchida A, Marumo K, Murai M, Activation Of Signal Transducer And Activator Of Transcription 3 In Renal Cell Carcinoma: A Study Of Indicence And Its Association With Pathological Features And Clinical Outcome, J Urology 2002; 168:762-765; Meinhold-Heerlein I, Bauerschlag D, Hilpert F, et al., Molecular And Prognostic Distinction Between Serous Ovarian Carcinomas Of Varying Grade And Malignant Potential, Oncogene 2005; 24:1053-1065; Masuda M, Suzui M, Yasumatu R, Constitutive Activation Of Signal Transducers And Activators Of Transcription 3 Correlates With Cyclin D1 Overexpression And May Provide A Novel Prognostic Marker In Head And Neck Squamous Cell Carcinoma, Cancer Res 2002; 62:3351-3355; Shah N G, Trivedi T I, Tankshali R A, STAT3 Expression In Oral Squamous Cell Carcinoma: Association With Clinicopathological Parameters And Survival, Int J Biol Markers 2006; 21:175-183; Yang S, Wang S, Wu C, et al., Altered p-STAT3 (tyr705) Expression Is Associated With Histological Grading And Intratumour Microvessel Density In Hepatocellular Carcinoma, J Clin Pathol 2007; 60:642-648; Khoury J D, Medeiros L J, Rassidakis G, et al., Differential Expression And Clinical Significance Of Tyrosine-Phosphorylated STAT3 In ALK+And ALK-Anaplastic Large Cell Lymphoma, Clin Cancer Res 2003; 9:3692-3699; Schlette E, Medeiros L J, Goy A, Lai R, Rassidakis G, Surviving Expression Predicts Poorer Prognosis In Anaplastic Large-Cell Lymphoma, J Clin Oncol 2004; 22:1682-1688; Lassmann S, Schuster I, Walch A, et al., STAT3 mRNA And Protein Expression In Colorectal Cancer Effects On STAT3-Inducible Targets Linked To Cell Survival And Proliferation, J Clin Pathol 2007; 60:173-179; Kusaba T, Nakayama T, Yamazumi K, et al., Expression Of p-STAT3 In Human Colorectal Adenocarcinoma And Adenoma; Correlation With Clinicopathological Factors, J Clin Pathol 2005; 58:833-838.

While these studies have addressed p-STAT3 phosphorylation at tyrosine⁷⁰⁵, others have shown that phosphorylation of p-STAT3 at the serine 727 location correlates with the degree of cervical intraepithelial neoplasia. Yang S, Yuan S, Yeh Y, et al., The Role Of p-STAT3 (ser727) Revealed By Its Association With Ki-67 In Cervical Intraepithelial Neoplasia, Gynecologic Oncol 2005; 98:446-452.

A study by Xie et al. confirms the importance of STAT3 in the process of metastasis. Xie T X, Wei D, Liu M, et al., Stat3 Activation Regulates The Expression Of Matrix Metalloproteinase-2 And Tumor Invasion And Metastasis, Oncogene 2004; 23:3550-3560. As shown, only highly metastatic melanoma cell lines overexpress MMP-2 and have elevated levels of p-STAT3. Furthermore, blockade of activated STAT3 by expression of dominant-negative STAT3 significantly suppressed MMP-2 expression and the invasiveness of melanoma cells, inhibited tumor growth, and prevented metastasis in nude mouse model systems. Therefore it is clearly established that STAT3 activation plays an important role in the dysregulated expression of basic fibroblast growth factor, VEGF, and MMP-2 and confirmed its effects on angiogenesis and its contribution to brain metastasis in melanoma. Subsequent studies of human melanoma cases have demonstrated higher levels of expression of activated STAT3 in brain metastasis specimens than in primary, parenchymal tumors. Xie T X, Huang F J, Aldape K D, et al., Activation Of Stat3 In Human Melanoma Promotes Brain Metastasis, Cancer Res 2006; 66:3188-3196.

Studies using decoy anti-sense STAT3 oligonucleotides and dominant-negative vectors have provided further convincing evidence that STAT3 is highly relevant to the growth and survival of several tumor types, including melanoma, in vitro and in vivo. Tang G S, Cai J M, Ni J, et al., Effects Of STAT3 Antisense Oligodeoxynucleotides On Apoptosis And Proliferation Of Mouse Melanoma Cell Line B16, Ai Zheng 2006; 25:269-274; Leong P L, Andrews G A, Johnson D E, et al., Targeted Inhibition Of Stat3 With A Decoy Oligonucleotide Abrogates Head And Neck Cancer Cell Growth, Proc Natl Acad Sci USA 2003; 100:4138-4143;

Thus it is clear that STAT3, drives the fundamental components of tumor malignancy and metastases in many parts of the body including the Central Nervous System (“CNS”). STAT3 promotes tumorigenesis by enhancing proliferation, angiogenesis, invasion, metastasis, and immunosuppression.

The compounds disclosed herein may inhibit Stat3 compounds by modes of inhibition of STAT3 function processes including dimerization, phosphorylation, DNA bindings, transcriptional activity, compete against endogenous DNA cis-element, and combinations thereof.

While not limited to any particular mechanism, it is believed that these active agents may inactivate one or more of six Signal Transducers and Activators of Transcription (STAT) pathways. For example, STAT3 inhibitors may remedy disease states with complete effectiveness. Kaposi's Sarcoma is one condition caused by activated Stat3 compounds. STATS or its pathway or any other of the STAT1 through STATE proteins and their pathways may also be blocked by the active agent to treat the dermatologic, gynecologic, and genital affected regions. Further, interleukin 6 (IL-6) and interleukin 9 (IL-9) signaling may also be affected by the active agent during treatment. Also, the caffeic acid compounds described herein may be useful in inhibiting cyclooxygenase-II enzyme (COX-2) associated with inflammation and pain. The caffeic acid compounds described herein may be useful in inhibiting the Janus kinase 2/Signal transducers (JAX2) which is believed to inhibit tumor cell growth and increases sensitivity to apoptotic stimuli, and thus, likely represent potential therapeutics for cancer therapy.

Many different dermatological, gynecological, and genital disorders relate to persistent inflammation and abnormal cell growth and proliferation. For example, lichen sclerosus forms pruritic white patches on the skin and may form scarring in and around the genital region, and lichen planus forms papules on the skin and oral mucosa of affected patients. The patches and papules of these conditions make the disorders both a medical and a cosmetic problem. Additionally, both conditions are related to abnormal cell growth and skin discoloration and are believed to increase risk of cancer in affected individuals.

There are also additional dermatological, gynecological, and genital disorders involving proliferative cell growth, such as a dysplasias and cancers of the vulva, vagina, and cervix, which fail to have satisfactory treatment—this applies primarily to dysplasias—such as treatments that are invasive and has consequences such as scarring, physical disfigurement, skin thinning, preterm labor and preterm birth, and smucositis from topical applications among other undesired side-effects. Patients may achieve only some improvement in their condition over long periods of several months, may have to undergo disfiguring surgery or chronically apply ultrapotent steroids.

Additional cell proliferative diseases that may be treated by administrating the compounds and compositions described herein which may include dermatitis and mucositis conditions of gynecological and genital regions including inflammatory dermatologic conditions, for example, lichen sclerosus, lichen planus, vaginal dysplasia, vaginal carcinoma, vulvar dysplasia, vulvar carcinoma, cervical dysplasia, cervical carcinoma, and Kaposi's sarcoma. Kaposi's sarcoma is a malignancy primarily of the skin but also other tissues wherein the etiologic virus is known to activate STAT3. Since the majority of the lesions are cutaneous/mucosal, topical agents would be well received. Additionally, Kaposi's sarcoma and the above mentioned gynecologic cancers, which are largely genital human papillomavirus (HPV) related, are much more prevalent in the immunocompromised population (HIV, organ transplant recipients, individuals with rheumatologic disorders on immunosuppresants etc). Both cervical cancer and Kaposi's sarcoma are considered AIDS defining illnesses.

### SUMMARY OF THE INVENTION

Novel compounds and pharmaceutical compositions, certain of which have been found to inhibit STAT3 have been discovered, together with methods of synthesizing and using the compounds including methods for the treatment of STAT3-mediated diseases in a patient by administering the compounds.

In certain embodiments of the present invention, disclosed herein is a method for treating a disease selected from the group consisting of lichen sclerosus, lichen planus, vaginal dysplasia, vaginal carcinoma, vulvar dysplasia, vulvar carcinoma, cervical dysplasia, and Kaposi's sarcoma, comprising the administration of a therapeutically effective amount of a compound having structural Formula I:

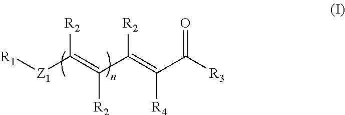

or a pharmaceutically acceptable salt thereof, wherein:

n is 0 or 1;

m is and integer selected from 1, 2, 3, or 4;

R₁ is selected from the group consisting of:

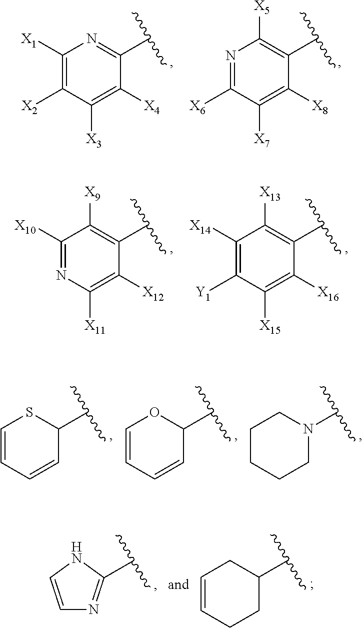

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

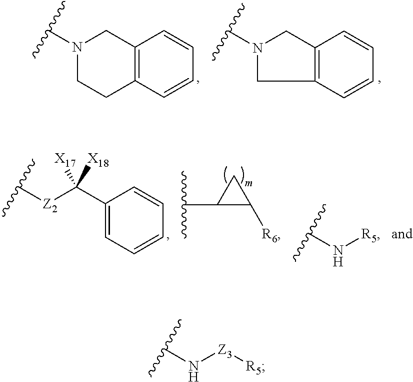

R₄ is selected from the group consisting of cyano, alkylamine, CH₂S-alkyl, alkyl, and CH₂N₃;

R₅ and R₆ are each independently selected from the group consisting of:

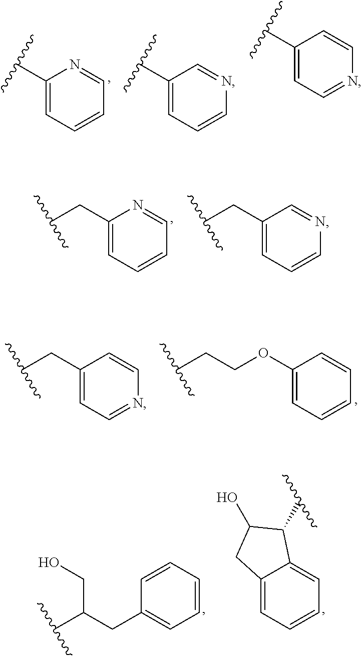

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁ᵢ, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of NH, S, and O; and

Z₃ is alkyl.

Certain compounds disclosed herein may possess useful STAT3 inhibiting activity, and may be used in the treatment or prophylaxis of a disease or condition in which STAT3 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting STAT3. Other embodiments provide methods for treating a STAT3-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of STAT3.

In certain embodiments, R₁ is selected from the group consisting of:

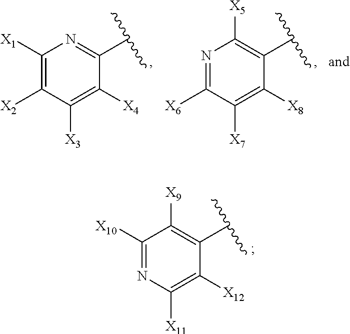

each instance of R₂ is hydrogen;

R₃ is

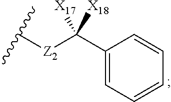

and

Z₂ is NH.

In further embodiments, X₁, X₂, X₃, X₄, X₅, X₆, X₇, X₈, X₉, X₁₀, X₁₁, and X₁₂ are each independently selected from the group consisting of hydrogen and halogen; and X₁₇ and X₁₈ are each independently selected from the group consisting of hydrogen, alkyl, and cycloalkyl.

In further embodiments,

R₁ is

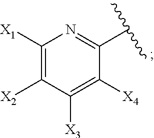

X₁ is halogen; and

X₂, X₃, and X₄ are hydrogen.

In further embodiments, one of X₁₇ and X₁₈ is hydrogen; the other of one of X₁₇ and X₁₈ is selected from the group consisting of hydrogen, methyl, ethyl, and cyclopropyl.

In further embodiments, n is 0.

In further embodiments, n is 1.

In further embodiments, the STAT3 inhibitor is selected from the group consisting of examples 1-65.

In further embodiments, the STAT3 inhibitor is:

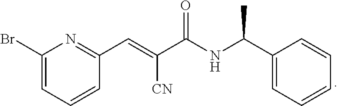

In further embodiments, the STAT3 inhibitor is:

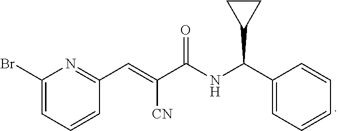

In further embodiments, the STAT3 inhibitor is:

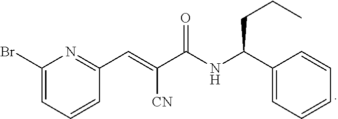

In further embodiments, the STAT3 inhibitor is:

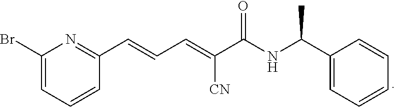

In further embodiments, the STAT3 inhibitor is:

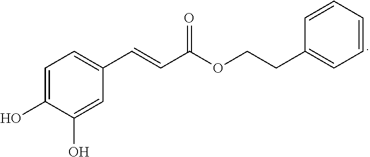

In further embodiments, the STAT3 inhibitor is:

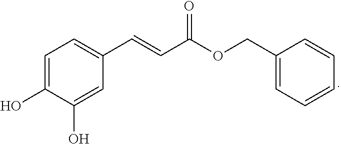

In further embodiments, the disease is lichen sclerosus.

In further embodiments, the disease is lichen planus.

In further embodiments, the disease is vaginal dysplasia.

In further embodiments, the disease is vaginal carcinoma.

In further embodiments, the disease is vulvar dysplasia.

In further embodiments, the disease is vulvar carcinoma.

In further embodiments, the disease is cervical dysplasia.

In further embodiments, the disease is Kaposi's sarcoma.

In further embodiments, the STAT3 inhibitor is administered as a pharmaceutical composition comprising the compound of Formula I at a concentration by weight within a range from about 0.01% to about 20% or or at a patient weight dosage within a range from about 1 mg/kg to about 100 mg/kg, together with a pharmaceutically acceptable carrier.

In further embodiments, the STAT3 inhibitor is administered as a pharmaceutical composition comprising the compound of Formula I at a concentration by weight within a range from about 1% to about 10% or or at a patient weight dosage within a range from about 1 mg/kg to about 60 mg/kg, together with a pharmaceutically acceptable carrier.

In further embodiments, the pharmaceutical composition is an oral or parenteral pharmaceutical composition.

In further embodiments, the pharmaceutical composition is a topical pharmaceutical composition.

In further embodiments, the topical pharmaceutical composition further comprises petroleum jelly or dimethyl sulfoxide.

In further embodiments, the topical pharmaceutical composition further comprises at least one compound selected from the group consisting of cell differentiating agents, anti-proliferative agents, mitochondrial inhibitors, topical steroids, immunosuppressive compounds, JAK2 inhibitors, JAK3 inhibitors, parathyroid hormone-related protein agonists, cell adhesion blockers, derivatives thereof, and combinations thereof.

In further embodiments, the topical pharmaceutical composition further comprises a cell differentiating agent selected from at least one of retinoic acid, retinoic acid derivative, vitamin D, or vitamin D analog.

In certain embodiments, compounds have the chemical structure:

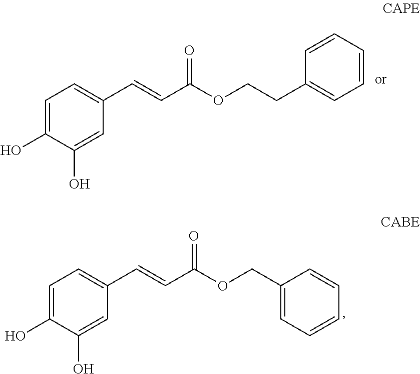

isomers thereof, derivatives thereof, pharmaceutical acceptable salts thereof, and combinations thereof.

In certain embodiments, compounds have the structure Formula II or structural Formula III:

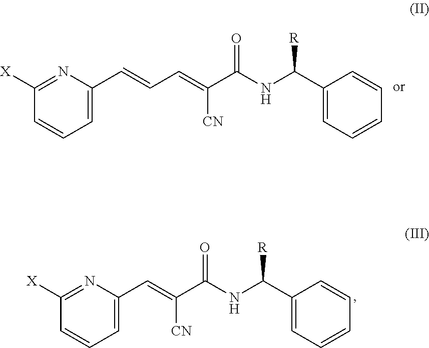

wherein X is a halogen and R is hydrogen, hydroxyl, alkyl, alkoxy, C₃-C₇-cycloalkyl, C₆-C₁₀-aryl, C₇-C₁₀-aralkyl, heteroatom-substituted or heteroatom-unsubstituted, isomers thereof, derivatives thereof, analogues, prodrugs, or pharmaceutical acceptable salts thereof.

In further embodiments, X may be bromide, and R may be methyl, ethyl, n-propyl, iso-propyl, cyclopropyl, n-butyl, sec-butyl, tert-butyl, as well as other alkyls, isomers thereof, or derivatives thereof.

In certain embodiments, compounds have the structure Formula IV or structural Formula V:

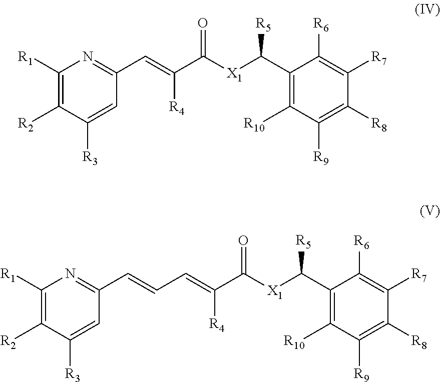

and pharmaceutically acceptable pharmaceutically acceptable salts thereof, where R₁, R₂, R₃, R₆, R₇, R₈, R₉, or R₁₀, are independently selected from H, OH, NO₂, N₃, NH₂, alkyl, alkoxy, O-acyl, COOH, F, Cl, Br, I, or derivatives thereof; R₄ is H, CN, or SO₂R; X₁ is O, NH, or S; and R₅ is H, alkyl, alkoxy, cycloalkyl, O-acyl, N-alkyl, N-acyl, or alkylamine. Alkyl groups usually have from 1 to 12 carbon atoms and include methyl, ethyl, propyl, butyl, amyl, isomers thereof, or derivatives thereof. In some examples, the alkyl groups may have from 1 to 6 carbon atoms. In other examples, the alkyl groups may have from 1 to 4 carbon atoms. The alkyl group may be optionally substituted with 1-3 substituents such as hydroxyl, alkylamine, —O-alkyl, acyl, —O-acyl, —N(R)-acyl, —C(O)—O-alkyl, —C(O)—N(R)-alkyl, thiol, and halo, where R is hydrogen or alkyl. Suitable alkyl groups include methyl, isopropyl, —CH₂-cyclohexyl, and cyclopropyl. In many examples, R₁ is a halogen or halide of fluorine, chlorine, bromine, or iodide.

In certain embodiments, a method for treating a dermatologic, gynecologic, and genital disorder is provided which includes applying a topical pharmaceutical composition to a lesion, wherein the topical pharmaceutical composition comprises at least one of the compounds disclosed herein.

In further embodiments, the topical pharmaceutical composition comprises the compound disclosed herein at a concentration by weight within a range from about 0.01% to about 20%, preferably, from about 0.1% to about 15%, preferably, from about 1% to about 10%, more preferably, from about 3% to about 7%, and more preferably, from about 4% to about 6%, for example, about 5%.

In some embodiments, the pharmaceutical composition may contain a compound disclosed herein at a concentration by weight within a range from about 0.01% to about 20% of the pharmaceutical composition or at a patient weight dosage within a range from about 1 mg/kg to about 100 mg/kg, preferably, from about 0.1% to about 15% or 1 mg/kg to about 60 mg/kg, preferably, from about 1% to about 10%, more preferably, from about 3% to about 7%, and more preferably, from about 4% to about 6%. In some examples, pharmaceutical compositions contain a compound disclosed herein at a concentration of about 5% by weight or 40 mg/kg. In one example, the topical pharmaceutical composition contains CAPE at a concentration of about 5% by weight. In another example, the topical pharmaceutical composition contains CABE at a concentration of about 5% by weight.

The pharmaceutical composition may further include a carrier suitable for topical administration, a carrier suitable for parenternal administration, or a carrier suitable for oral administration.

In another embodiment, a method for treating dermatologic, gynecologic, and genital disorders provides applying the topical pharmaceutical composition on lesions at various intervals, for example, at multiple times daily until symptoms of the disorder disappear.

In another embodiment, a method for treating dermatologic, gynecologic, and genital disorders provides applying the pharmaceutical composition by systemic administration.

### DETAILED DESCRIPTION

### Definitions

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the term “patient” in the context of the present invention is preferably a mammal. The mammal can be a human, non-human primate, mouse, rat, dog, cat, horse, or cow, but are not limited to these examples. Mammals other than humans can be advantageously used as patients that represent animal models of specific diseases and disorders. A patient can be male or female. A patient can be one who has been previously diagnosed or identified as having cellular degeneration or insufficiency, and optionally has already undergone, or is undergoing, a therapeutic intervention. Preferably the patient is human.

The terms “treating” or “treatment” means to relieve, alleviate, delay, reduce, reverse, improve, manage, or prevent at least one symptom of a condition in a patient. The term “treating” may also mean to arrest, delay the onset (i.e., the period prior to clinical manifestation of a disease), and/or reduce the risk of developing or worsening a condition.

“STAT3 inhibitor” is used herein to refer to a compound that exhibits an IC₅₀ with respect to STAT3 activity of no more than about 100 μM and more typically not more than about 50 μM, as measured in the STAT3 assay described generally hereinbelow. “IC₅₀” is that concentration of inhibitor which reduces the activity of an enzyme (e.g., STAT3) to half-maximal level. Certain compounds disclosed herein have been discovered to exhibit inhibition against STAT3. In certain embodiments, compounds will exhibit an IC₅₀ with respect to STAT3 of no more than about 10 μM; in further embodiments, compounds will exhibit an IC₅₀ with respect to STAT3 of no more than about 5 μM; in yet further embodiments, compounds will exhibit an IC₅₀ with respect to STAT3 of not more than about 1 μM; in yet further embodiments, compounds will exhibit an IC₅₀ with respect to STAT3 of not more than about 200 nM, as measured in the STAT3 assay described herein.

The term “Stat3,” as used herein, refers to any form of Stat3 known to those of skill in the art, including, but not limited to, Stat3α and Stat3β.

The term “proliferative disease,” as used herein, refers to any condition in which a localized population of proliferating cells in an animal is not governed by the usual limitations of normal growth. Examples of hyperproliferative disorders include tumors, neoplasms, lymphomas and the like and non-cancer disorders such as autoimmune diseases (e.g., psoriasis).

The term “neoplastic disease,” as used herein, refers to any abnormal growth of cells being either benign (non-cancerous) or malignant (cancerous). A neoplasm is said to be benign if it does not undergo invasion or metastasis and malignant if it does either of these.

As used herein, the terms “therapeutically effective amount”, “prophylactically effective amount”, or “diagnostically effective amount” is the amount of the active agent, e.g. STAT3 inhibitor, needed to elicit the desired biological response following administration.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C═C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′)— group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The terms “mercaptyl” or “mercaptan” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “monosaccharide” refers to a single basic sugar unit with the general formula Cₙ(H₂O)ₙ, with n ranging from 3 to 8. (e.g. glucose, fructose, galactose, stc.). Monosaccharides may form a glycosidic bond to another group to which they are attached, such as a hydroxyl group or an amino group.

The term “polysaccharide” refers to a polymeric group formed from two or more monosaccharides joined together by glycosidic bonds.

The term “monosaccharide derivative” refers to a monosaccharide that has been chemically modified by addition of one or more protecting groups, such as acetyl groups or diisopropylidene groups (e.g., acetylated galactose, 1,2,3,4-diisopropylideno-D-galactose, etc.).

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

In some embodiments, a topical pharmaceutical composition contains a compound as disclosed herein at a concentration by weight within a range from about 0.01% to about 20%, preferably, from about 0.1% to about 15%, preferably, from about 1% to about 10%, more preferably, from about 3% to about 7%, and more preferably, from about 4% to about 6%. In one example, the topical pharmaceutical composition contains a compound as disclosed herein at a concentration by weight of about 5%.

In other embodiments, the topical dosage forms may contain about 0.5% to about 20.0% by weight of a compound as disclosed herein. The weight percent of the compound described herein within the topical dosage forms may range from about 1.0% to about 15.0% or about 5.0% to about 11.0% by weight of the pharmaceutical composition. Treatment regimes with the topical dosage forms may occur daily, twice daily, three times daily, or four times daily for durations, for example, of three weeks or four weeks or until symptoms are no longer present. In some examples, the topical dosage contains a compound as disclosed herein at a concentration of about 5% by weight of the pharmaceutical composition. In other examples, the topical dosage contains a compound as disclosed herein at a concentration of about 5% by weight of the pharmaceutical composition.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with cell differentiating agents, anti-proliferative agents, mitochondrial inhibitors, topical steroids, immunosuppressive compounds, JAK inhibitors (including JAK2 and JAK3 inhibitors), tyrosine kinase inhibitors such as Src inhibitors and Scr derivatives/family inhibitors, epidermal growth factor receptor (EGFR) inhibitors/compounds, parathyroid hormone-related protein (PTHrP) agonists, interleukins IL-6 inhibitors, cytokine inhibitors such as SH2 inhibitors, cell adhesion blockers, and combinations thereof.

In certain embodiments, possible combination therapies include use of certain compounds of the invention with cell differentiating and anti-proliferative agents (e.g., retinoic acid, retinoids (tazarotene), vitamin D, or vitamin D analogs (calcipotriene)); mitochondrial inhibitors (e.g., anthraline (dithranol, chrysarobin, or coal tar)); topical steroids (e.g., clobetasol propionate, betamethasone, betamethasone dipropionate, halobetasol propionate, fluocinonide, diflorasone diacetate, mometasone furoate, halcinonide, desoximetasone, fluticasone propionate, flurandrenolide, triamcinolone acetonide, fluocinolone acetonide, hydrocortisone, hydrocortisone valerate, prednicarbate, desonide, or alclometasone dipropionate); immunosuppressive compounds (e.g., tacrolimus (FK-506)); JAK2 inhibitors (e.g., INCB18424); JAK3 inhibitors (e.g., CP-690,550); parathyroid hormone-related protein (PTHrP) agonists (e.g., PTH (1-34)); and cell adhesion blockers (e.g., pan-selectin antagonist bimosiamose).

In further embodiments, examples of cell differentiating agents include a retinoid, such as retinoic acid, vitamin D, vitamin D analogs, or a phorbol ester. The term vitamin D collectively refers to a group of structurally similar chemicals and their metabolites which include alfacalcidol (1-hydroxycholecalciferol), calcitriol (1,25-dihydroxycholecalciferol), cholecalciferol (vitamin D3), dihydrotachysterol (DHT) and ergocalciferol (vitamin D2). The active metabolite of vitamin D, 1,25-(OH)₂D₃, has a wide range of nonclassical actions in the body, such as regulation of cell growth and differentiation modulation of the immune system.

In certain embodiments, the compounds disclosed herein may tend to stop an active condition, for example, a lesion, condition, discoloration, or palpable tumor, while the cell differentiating agent may lessen the likelihood of reactivation of the active condition to prevent recurrence.

In certain embodiments, JAK inhibitors include one or more compounds selected from the group of MK-0457, CEP-701 (Lestaurtinib), Erlotinib (Tarceva), AT9283, TG101209, and Go6976. JAK2-selective inhibitors include INCB018424, XL019, TG101348, and combinations thereof. Non-JAK2 selective inhibitors MK-0457, CEP-701 (Lestaurtinib), AT9283, and combinations thereof. Other JAK/STAT3 inhibitors may include compounds selected from the group of cucurbitacin I, curcumin, magnolol, indirubin, resveratol, flavopriridol, galiellalacton, and combinations thereof.

In certain embodiments, combination therapies include use of certain compounds of the invention with oglionucleotides, such as decoy oligonucleotide abrogates. Examples of such oglionucleotides include Stat3 decoy and mutant control decoy oligonucleotides available from MWG Biotech of high Point North Carolina. Other oglionucleotides that may be used include siRNAs, G-Quartet oglionucleotides, dominant-negative mutant oglionucleotides, anti-sense approach oglionucleotides, and combinations thereof.

In certain embodiments, combination therapies include use of certain compounds of the invention with nonpeptidic small molecules. These compounds, such as Stattic, 6-nitro-benzo[b]thiophene-1,1-dioxide 1, (including benzo[b]thiophenesulphonamides) are shown to selectively inhibit the function of sTAT3 SH2 domain regardless of the activation state in vitro. Examples of nonpeptidic small molecules, further include STA-21, which is described in U.S. patent application Ser. No. 11/361,149 and PCT application PCT/US2006/006637, both of which are incorporated herein by reference to the extent not inconsistent with the description and claimed aspects herein.

In certain embodiments, combination therapies include use of certain compounds of the invention with steroids, such as topical steroids, for treatment of lichen sclerosus.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating STAT3-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of STAT3-mediated disorders.

Specific diseases to be treated by the compounds, compositions, and methods disclosed herein include those of the circulatory, digestive, endocrine, integument, muscular, nervous, reproductive, respiratory, skeletal and urinary systems. These diseases may be congenital in nature or relate to later onset. In addition, the compounds, compositions, and methods disclosed herein may be used to treat injury to those same organ systems.

Thus, in another aspect, certain embodiments provide methods for the promotion of dermal regeneration (wound healing) in a human or animal subject in need of such treatment comprising administering to said subject an amount of a compound disclosed herein effective to induce and enhance regeneration in the subject, in combination with at least one additional agent for the treatment of said disorder that is known in the art. In a related aspect, certain embodiments provide therapeutic compositions comprising at least one compound disclosed herein in combination with one or more additional agents for the treatment of acute surgical or traumatic wounds.

In certain embodiments, compounds and methods described herein may be used to treat a variety of dermatologic, gynecologic, and genital cancerous states including, for example, vulvar cancer, vaginal cancer, cervical cancer, and Kaposi's Sarcoma. The cancer may comprise a tumor made of cancer cells. These cancerous states may include cells that are cancerous (including in situ carcinoma), pre-cancerous (dysplasia), and/or malignant (neoplasia) including vaginal dysplasia, vaginal carcinoma, vulvar dysplasia, vulvar carcinoma, cervical dysplasia, cervical carcinoma, and combinations thereof. Other disorders, such as squamous cell carcinoma/cancers are contemplated as being treated with the caffeic acid compounds described herein. Additionally, Kaposi's Sarcoma is a condition associated with an HIV infection, the caffeic acid compounds disclosed herein may have some therapeutic benefit for HIV infected individuals.

In further embodiments, compounds and methods described herein may be used to treat cell proliferative diseases other than cancer. The caffeic acid compounds and methods described herein may also be used to treat dermatological, including mucosa (mucositis) conditions of the genital and oral regions, such as lichen sclerosus and lichen planus. Lichen sclerosus and lichen planus may lead to squamous cell carcinoma/cancers. The caffeic acid compounds may also be used for inflammatory dermatologic conditions.

In many embodiments, the pharmaceutical compositions containing the caffeic acid compounds (e.g., caffeic acid ester compounds or caffeic acid amide compounds) may be useful to treat various tumors which include solid or palpable tumors, such as Kaposi's scarnoma.

In certain embodiments, therapeutic benefits of the compounds disclosed herein include extension of the patient's life by any period of time; decrease or delay in the neoplastic development of the disease; decrease in hyperproliferation; reduction in tumor growth; delay of metastases; reduction in the proliferation rate of a cancer cell, tumor cell, or any other hyperproliferative cell; induction of apoptosis in any treated cell or in any cell affected by a treated cell; and/or a decrease in pain to the subject that may be attributed to the patient's condition.

In certain embodiments, the compounds disclosed herein can be used in combination with surgery, chemotherapy, radiotherapy, and/or a gene therapy. For example, the compounds could be applied topically or systemically with traditional chemotheraphy application methods. Additional, the compounds and any chemotherapy compounds may be combined and used in a chemitopical application or intravenous solution.

In practice, the compounds, compositions, and methods disclosed herein include the use as part of a medical device. The medical device can be designed for implantation into the body or can be used to function outside of the body as an approach to treating the diseases and injuries described above. When used outside of the body, the device containing compounds, compositions, and methods disclosed herein would be attached to the appropriate site on the body in order to carry out the prescribed function.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3, 4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth.

Synthetic Chemistry. The compounds disclosed herein can be prepared by following the procedures described in WO 2005058829 (pages 22-29), US 20050277680, U.S. Pat. No. 7,745,468 (column 20, line 1 to column 25, line 12), WO 2007115269 (pages 40-52), US 20070232668 (pages 17-22), and WO 2010005807 (paragraphs [0191]-[0201]), each of which is hereby incorporated by reference in their entirety; methods known to one of skill in the art; and routine modifications thereof.

Compounds described herein may be synthesized by utilizing various methods of ester formation which leads to formation of an ester bond between caffeic acid and its analogs and respective alcohols. For example, Fischer Esterification (Fischer-Speier Esterification) utilizes a Lewis or Brønstedt acid catalyzed esterification of carboxylic acids with alcohols to give esters via a reaction in which the products and reactants are in equilibrium, as may be influenced by either removing one product from the reaction mixture (for example, removal of the water by azeotropic distillation or absorption by molecular sieves) or by employing an excess of one reactant. Alternative reactions employ coupling reagents such as dicyclohexylcarbodiimide (Steglich Esterification), preformed esters (transesterification), carboxylic acid chlorides or anhydrides. Esters may also be produced by oxidations, such as by the Baeyer-Villiger oxidation and oxidative esterifications. Similar procedures may be used to make the analogs.

The following reaction illustrates synthesis of caffeic acid phenyl ester

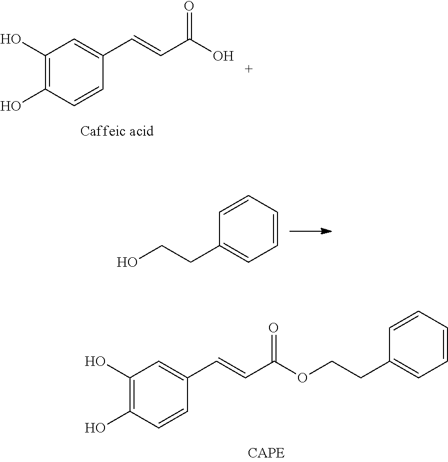

The invention is further illustrated by the following examples. All IUPAC names were generated using CambridgeSoft's ChemDraw 11.0.

### Example 1

### (S,E)-3-(6-bromopyridin-2-yl)-2-cyano-N-(1-phenylethyl)acrylamide (WP1066)

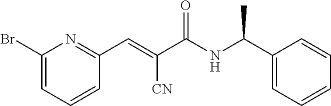

### Example 2

### (E)-N-benzyl-3-(6-bromopyridin-2-yl)-2-cyanoacrylamide (WP1015)

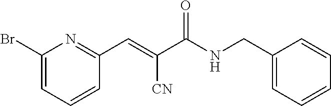

### Example 3

### (S,E)-3-(6-bromopyridin-2-yl)-2-cyano-N-(1-phenylbutyl)acrylamide (WP1130)

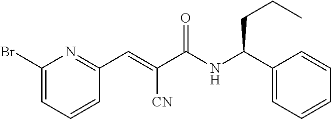

### Example 4

### (S,E)-3-(6-bromopyridin-2-yl)-2-cyano-N-(1-phenylpropyl)acrylamide (WP1129)

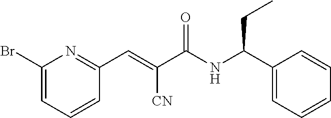

### Example 5

### (E)-N-benzyl-2-cyano-3-(4-nitrophenyl)acrylamide (AG1801)

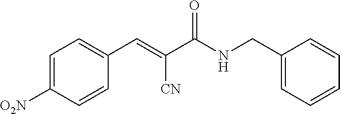

### Example 6

### (R,E)-2-cyano-3-(4-nitrophenyl)-N-(1-phenylethyl)acrylamide (WP1034)

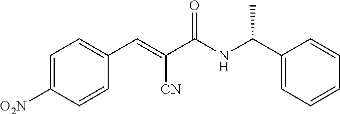

### Example 7

### (R,E)-2-cyano-3-(3-hydroxy-4-nitrophenyl)-N-(1-phenylethyl)acrylamide (WP1038)

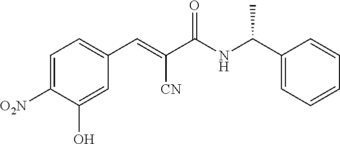

### Example 8

### (S,E)-2-cyano-3-(4-nitrophenyl)-N-(1-phenylethyl)acrylamide (WP1050)

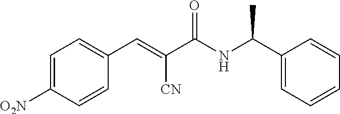

### Example 9

### (S,E)-2-cyano-3-(3-hydroxy-4-nitrophenyl)-N-(1-phenylethyl)acrylamide (WP1051)

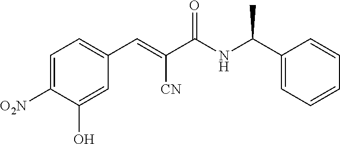

### Example 10

### (S,E)-2-cyano-N-(1-phenylethyl)-3-(pyridin-2-yl)acrylamide (WP1065)

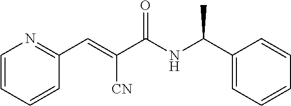

### Example 11

### (E)-N-benzyl-2-cyano-3-(pyridin-3-yl)acrylamide (WP1075)

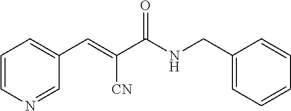

### Example 12

### (E)-N-benzyl-3-(4-chloro-3-nitrophenyl)-2-cyanoacrylamide (WP1077)

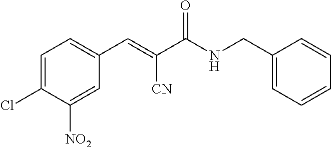

### Example 13

### (S,E)-cyclopropyl(phenyl)methyl 3-(6-bromopyridin-2-yl)-2-cyanoacrylate (WP1332)

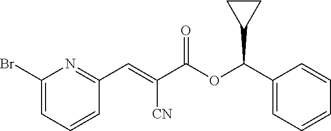

### Example 14

### (S,E)-2-cyano-N-(cyclopropyl(phenyl)methyl)-3-(3,4-dihydroxyphenyl)acrylamide (WP1331)

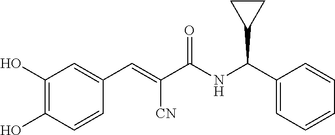

### Example 15

### (S,E)-cyclopropyl(phenyl)methyl 2-cyano-3-(3,4-dihydroxyphenyl)acrylate (WP1330)

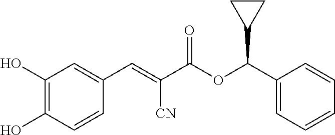

### Example 16

### (S,E)-cyclopropyl(phenyl)methyl 3-(3,4-dihydroxyphenyl)acrylate (WP1329)

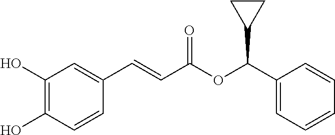

### Example 17

### (S,E)-N-(cyclopropyl)phenyl)methyl)-3-(3,4-dihydroxyphenyl)acrylamide (WP1328)

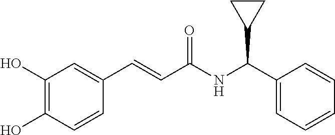

### Example 18

### (R,E)-2-(3-(6-bromopyridin-2-yl)-2-cyanoacrylamido)-2-phenylethyl pivalate (WP1302)

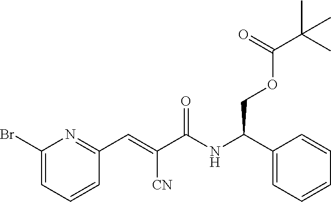

### Example 19

### (R,E)-2-(3-(6-bromopyridin-2-yl)-2-cyanoacrylamido)-2-phenylethyl acetate (WP1293)

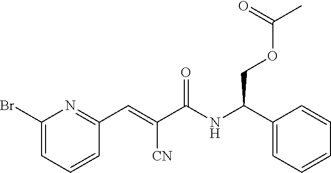

### Example 20

### (S,E)-3-(6-bromopyridin-3-yl)-2-cyano-N-(1-phenylethyl)acrylamide (WP1286)

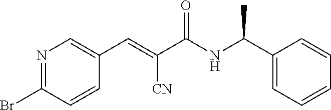

### Example 21

### (S,E)-3-(6-bromopyridin-2-yl)-N-(1-phenylethyl)acrylamide (WP1204)

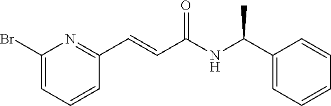

### Example 22

### (S,E)-3-(2-bromopyridin-3-yl)-2-cyano-N-(1-phenylethyl)acrylamide (WP1285)

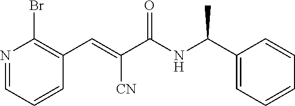

### Example 23

### (S,E)-3-(5-bromopyridin-3-yl)-2-cyano-N-(1-phenylethyl)acrylamide (WP1284)

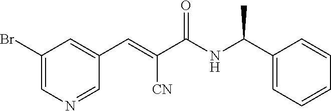

### Example 24

### (S,E)-3-(3-bromopyridin-4-yl)-2-cyano-N-(1-phenylethyl)acrylamide (WP1283)

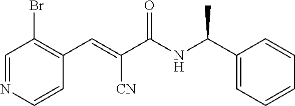

### Example 25

### (S,E)-3-(6-bromopyridin-2-yl)-2-cyano-N-(cyclopentyl(phenyl)methyl)acrylamide (WP1282)

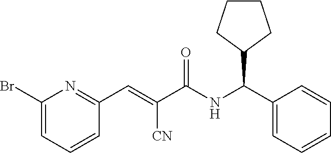

### Example 26

### (S,E)-2-cyano-3-(3-fluoropyridin-4-yl)-N-(1-phenylethyl)acrylamide (WP1280)

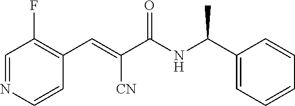

### Example 27

### ((S,E)-2-cyano-3-(2-methoxypyridin-3-yl)-N-(1-phenylethyl)acrylamide (WP1273)

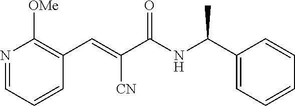

### Example 28

### (S,E)-2-cyano-N-(cyclopropyl(phenyl)methyl)-3-(2-fluoropyridin-3-yl)acrylamide (WP1272)

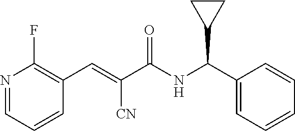

### Example 29

### (S,E)-(6-(2-cyano-3-(cyclopropyl(phenyl)methylamino)-3-oxoprop-1-enyl)pyridin-2-yl)methyl acetate (WP1246)

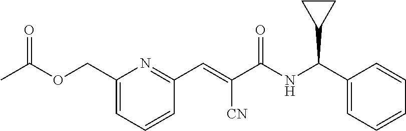

### Example 30

### (S,E)-3-(6-chloropyridin-2-yl)-2-cyano-N-(cyclopropyl(phenyl)methyl)acrylamide (WP1229)

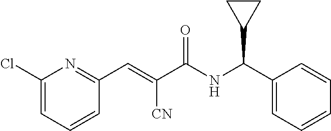

### Example 31

### (R,E)-3-(6-bromopyridin-2-yl)-2-cyano-N-(2-hydroxy-1-phenylethyl)acrylamide (WP1269)

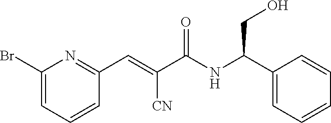

### Example 32

### (S,E)-2-cyano-3-(2-fluoropyridin-3-yl)-N-(1-phenylethyl)acrylamide (WP1271)

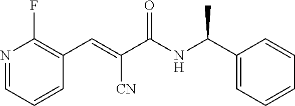

### Example 33

### (S,E)-3-(6-bromopyridin-2-yl)-2-cyano-N-(1-hydroxy-3-phenylpropan-2-yl)acrylamide (WP1268)

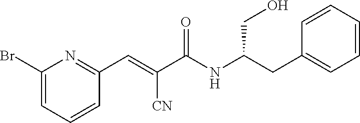

### Example 34

### (S,E)-3-(6-bromopyridin-2-yl)-2-cyano-N-(cyclobutyl(phenyl)methyl)acrylamide (WP1267)

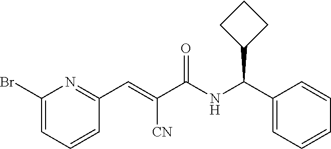

### Example 35

### (S,E)-2-cyano-3-cyclododecyl-N-(1-phenylethyl)acrylamide (WP1203)

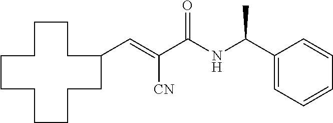

### Example 36

### (S,E)-2-(6-bromopyridin-2-yl)-N-(1-phenylethyl)ethenesulfonamide (WP1201)

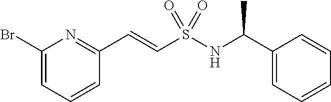

### Example 37

### (S,E)-2-cyano-3-(1H-imidazol-2-yl)-N-(1-phenylethyl)acrylamide (WP1196)

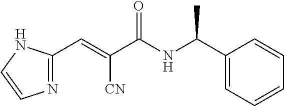

### Example 38

### (R,E)-2-cyano-3-(6-methylpyridin-2-yl)-N-(1-phenylethyl)acrylamide (WP1180)

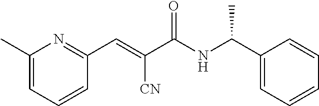

### Example 39

### (S,E)-2-cyano-3-(6-methylpyridin-2-yl)-N-(1-phenylethyl)acrylamide (WP1179)

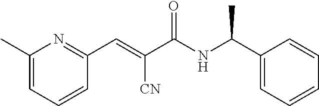

### Example 40

### (E)-N-benzhydryl-3-(6-bromopyridin-2-yl)-2-cyanoacrylamide (WP1169)

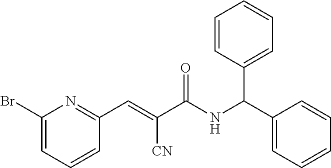

### Example 41

### (E)-3-(6-bromopyridin-2-yl)-2-cyano-N-((1R,2S)-2-hydroxy-2,3-dihydro-1H-inden-1-yl)acrylamide (WP1168)

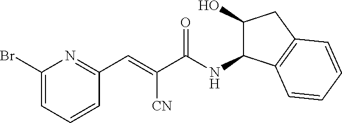

### Example 42

### (E)-3-(6-bromopyridin-2-yl)-2-cyano-N-((1S,2R)-2-hydroxy-2,3-dihydro-1H-inden-1-yl)acrylamide (WP1167)

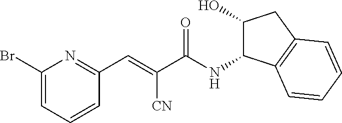

### Example 43

### (S,E)-3-(6-bromopyridin-2-yl)-2-cyano-N-(cyclohexyl(phenyl)methyl)acrylamide (WP1166)

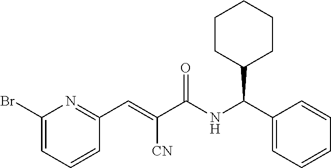

### Example 44

### (S,E)-3-(6-bromopyridin-2-yl)-2-cyano-N-(cyclobutyl(phenyl)methyl)acrylamide (WP1164)

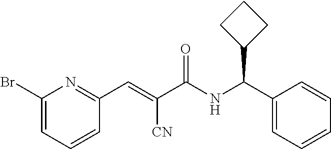

### Example 45

### (R,E)-3-(6-bromopyridin-2-yl)-2-cyano-N-(cyclopropyl(phenyl)methyl)acrylamide (WP1163)

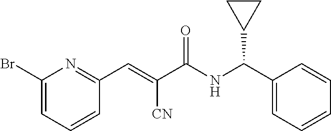

### Example 46

### (E)-3-(6-bromopyridin-2-yl)-2-cyano-N-(2-phenoxyethyl)acrylamide (WP1159)

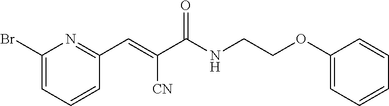

### Example 47

### (S,E)-3-(6-bromopyridin-2-yl)-2-cyano-N-(1,2-diphenylethyl)acrylamide (WP1145)

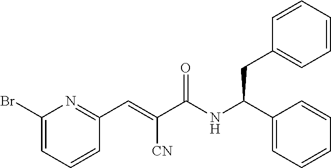

### Example 48

### (S,E)-3-(6-bromopyridin-2-yl)-2-cyano-N-(cyclopropyl(phenyl)methyl)acrylamide (WP1193)

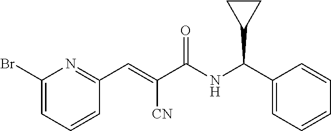

### Example 49

### (E)-N-benzyl-2-cyano-3-(cyclohex-3-enyl)acrylamide (WP1082)

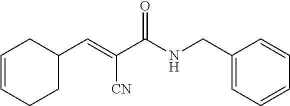

### Example 50

### (2E,4E)-5-(6-bromopyridin-2-yl)-2-cyano-N—((S)-1-phenylethyl)penta-2,4-dienamide (WP1220)

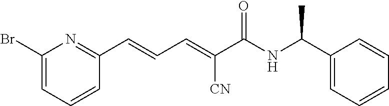

### Example 51

### (2E,4E)-5-(6-chloropyridin-2-yl)-2-cyano-N—((S)-1-phenylethyl)penta-2,4-dienamide

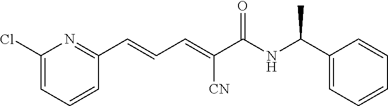

### Example 52

### (2E,4E)-5-(6-bromopyridin-2-yl)-2-cyano-N—((R)-1-phenylethyl)penta-2,4-dienamide

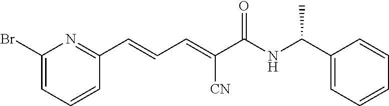

### Example 53

### (2E,4E)-N-benzyl-5-(6-bromopyridin-2-yl)-2-cyanopenta-2,4-dienamide

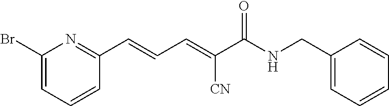

### Example 54

### (2E,4E)-5-(6-chloropyridin-2-yl)-2-cyano-N—((R)-1-phenylethyl)penta-2,4-dienamide

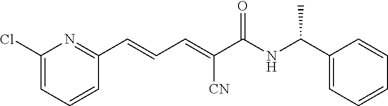

### Example 55

### (2E,4E)-N-benzyl-5-(6-chloropyridin-2-yl)-2-cyanopenta-2,4-dienamide

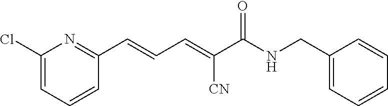

### Example 56

### (2E,4E)-5-(6-bromopyridin-2-yl)-2-cyano-N—((S)-cyclopropyl(phenyl)methyl)penta-2,4-dienamide

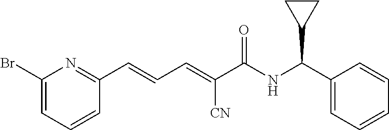

### Example 57

### (2E,4E)-5-(6-bromopyridin-2-yl)-2-cyano-N—((S)-1-phenylpropyl)penta-2,4-dienamide

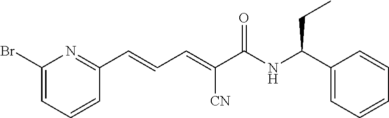

### Example 58

### (2E,4E)-5-(6-bromopyridin-2-yl)-2-cyano-N—((S)-1-phenylbutyl)penta-2,4-dienamide

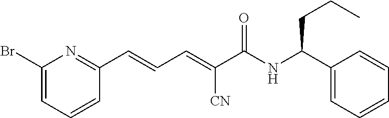

### Example 59

### (E)-N-benzyl-2-cyano-3-(3,4-dihydroxyphenyl)acrylamide (AG490)

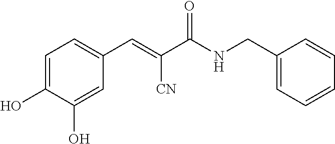

### Example 60

### (E)-N-benzyl-2-cyano-3-(5-hydroxy-2-nitrophenyl)acrylamide (WP1073)

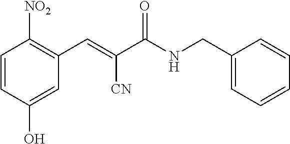

### Example 61

### (E)-N-benzyl-2-cyano-3-(3-nitrophenyl)acrylamide (WP1074)

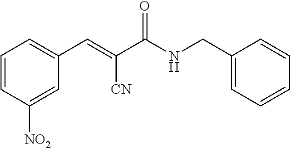

### Example 62

### (E)-N-benzyl-2-cyano-3-(4-(dimethylamino)-2-nitrophenyl)acrylamide (WP1076)

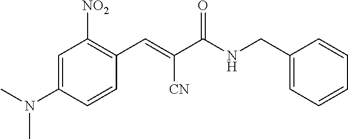

### Example 63

### (E)-3-(6-bromopyridin-2-yl)-2-cyano-N-4(2R,3R,4S,5R,6S)-3,4,5,6-tetrahydroxytetrahydro-2H-pyran-2-yl)methyl)acrylamide (WP1126)

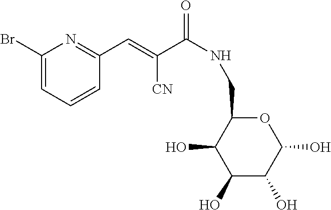

### Example 63

### (E)-3-(6-bromopyridin-2-yl)-2-cyano-N-4(3aR,5R,5aS,8aS,8bR)-2,2,7,7-tetramethyltetrahydro-3aH-bis[1,3]dioxolo[4,5-b:4′,5′-d]pyran-5-yl)methyl)acrylamide (WP1119)

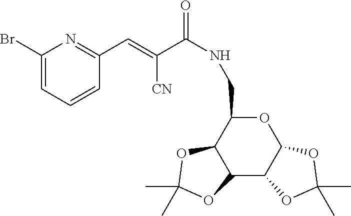

### Example 63

### (2R,3R,4S,5S,6R)-6-(((E)-3-(6-bromopyridin-2-yl)-2-cyanoacrylamido)methyl)tetrahydro-2H-pyran-2,3,4,5-tetrayl tetraacetate (WP1127)

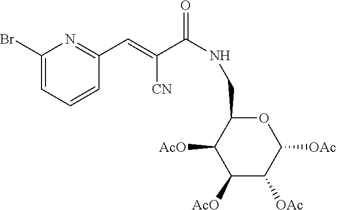

### Example 64

### (E)-Phenethyl 3-(3,4-dihydroxyphenyl)acrylate (CAPE)

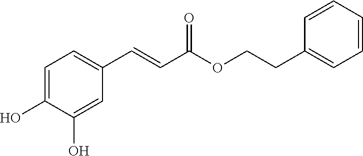

### Example 65

### (E)-Benzyl 3-(3,4-dihydroxyphenyl)acrylate (CABE)

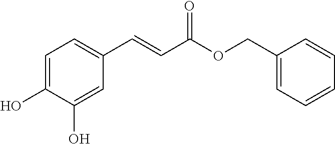

### Example 66

Cellular Assays—Certain compounds disclosed herein have been tested and found to be active in various cellular assays of STAT3 inhibition, cytotoxicity, or anti-cancer activity. For example, see WO 2005058829 (pages 29-34 and FIGS. 1-3 and 5-7), U.S. Pat. No. 7,745,468 (column 25, line 14 to column 31, line 44, and FIGS. 1-3 and 5-7), US 20070232668 (pages 22-23 and FIGS. 1-34), WO 2007115269 (pages 52-54 and FIGS. 1-34), WO 2010005807 (pages 42-43 and FIGS. 1-4), each of which is hereby incorporated by reference.

### Example 67

In Vivo Human A375 Melanoma Assay—Certain compounds disclosed herein have been tested and found to decrease tumor size in human A375 melanoma tumors grown in nude mice. For example, see WO 2005058829 (pages 34-35 and FIG. 8), U.S. Pat. No. 7,745,468 (column 25, line 14 to column 31, line 44, and FIG. 8), each of which is hereby incorporated by reference.

### Example 68

Human Psoriasis Study of CAPE—Certain compounds disclosed herein have been tested and found to reduce and/or resolve psoratic lesions in humans. For example, see US 20080167277 (page 3 and FIGS. 1-5), WO 2008083389 (page 9 and FIGS. 1-5), each of which is hereby incorporated by reference.

### Example 69

Human Psoriasis Study of WP1220 and CABE—The compounds WP1220 and CABE were tested in a pilot study of psoriasis patients who were not responsive to current standard therapies. After topical treatment with WP1220, seven out of seven patients responded with substantial improvement or complete resolution of psoratic lesions. After topical treatment with CABE, five out of seven patients responded with substantial improvement or complete resolution of psoratic lesions.

What is claimed is:

1. A method for treating a disease selected from the group consisting of lichen sclerosus, lichen planus, vaginal dysplasia, vaginal carcinoma, vulvar dysplasia, vulvar carcinoma, cervical dysplasia, and Kaposi's sarcoma, comprising the administration of a therapeutically effective amount of a compound having structural Formula I: 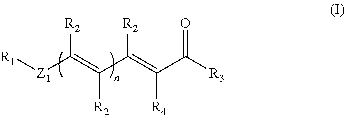
or a pharmaceutically acceptable salt thereof, wherein:
n is 0 or 1;
m is and integer selected from 1, 2, 3, or 4;
R₁ is selected from the group consisting of: 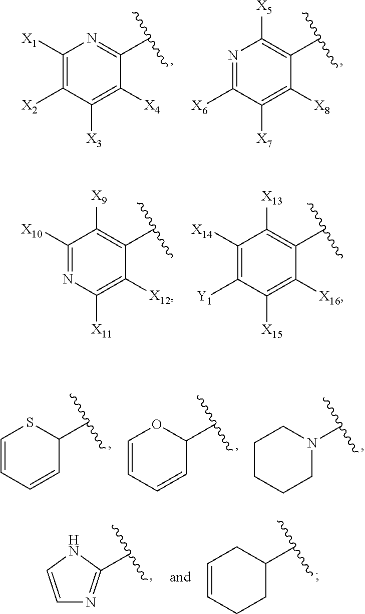
each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;
R₃ is selected from the group consisting of: 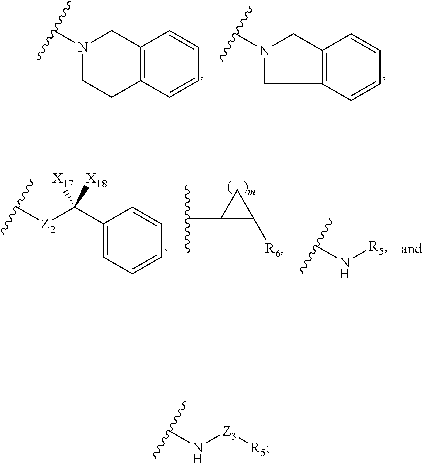
R₄ is selected from the group consisting of cyano, alkylamine, CH₂S-alkyl, alkyl, and CH₂N₃;
R₅ and R₆ are each independently selected from the group consisting of: 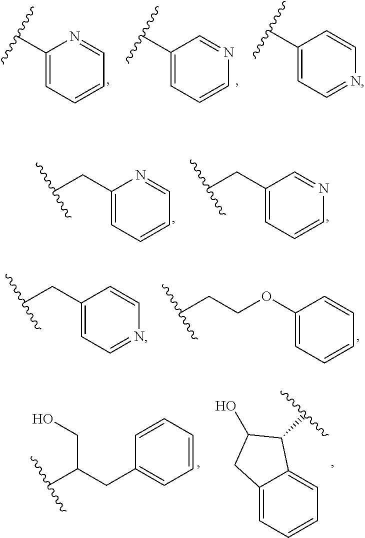
monosaccharide, polysaccharide, monosaccharide derivative, optionally substituted aryl, and optionally substituted arylalkyl;
X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;
X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;
Y₁ is selected from the group consisting of hydroxyl, halogen, and nitro;
Z₁ is selected from the group consisting of alkyl and a bond;
Z₂ is selected from the group consisting of NH, S, and O; and
Z₃ is alkyl.

2. The method of claim 1, wherein:
R₁ is selected from the group consisting of: 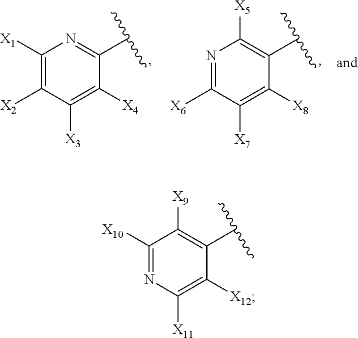
each instance of R₂ is hydrogen;
R₃ is 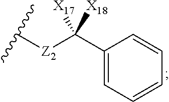
and
Z₂ is NH.

3. The method of claim 2, wherein:
X₁, X₂, X₃, X₄, X₅, X₆, X₇, X₈, X₉, X₁₀, X₁ᵢ, and X₁₂ are each independently selected from the group consisting of hydrogen and halogen; and
X₁₇ and X₁₈ are each independently selected from the group consisting of hydrogen, alkyl, and cycloalkyl.

4. The method of claim 3, wherein:
R₁ is 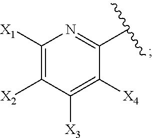
X₁ is halogen; and
X₂, X₃, and X₄ are hydrogen.

5. The method of claim 4, wherein:
one of X₁₇ and X₁₈ is hydrogen;
the other of one of X₁₇ and X₁₈ is selected from the group consisting of hydrogen, methyl, ethyl, and cyclopropyl.

6. The method of claim 5, wherein n is 0.

7. The method of claim 5, wherein n is 1.

8. The method of claim 1, wherein the STAT3 inhibitor is selected from the group consisting of examples 1-65.

9. The method of claim 1, wherein the compound has the structural formula:

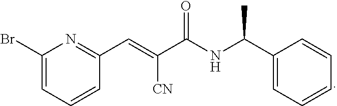

10. The method of claim 1, wherein the compound has the structural formula:

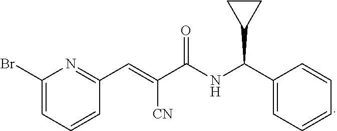

11. The method of claim 1, wherein the compound has the structural formula:

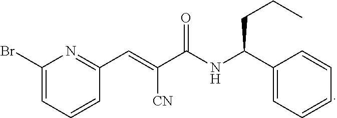

12. The method of claim 1, wherein the compound has the structural formula:

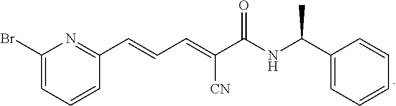

13. The method of claim 1, wherein the compound has the structural formula:

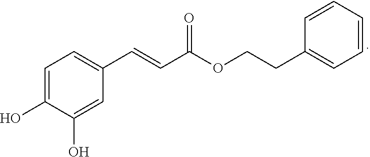

14. The method of claim 1, wherein the compound has the structural formula:

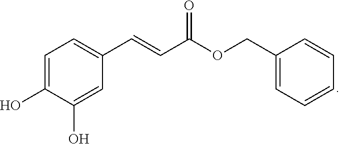

15. The method of claim 1, wherein the disease is lichen sclerosus.

16. The method of claim 1, wherein the disease is lichen planus.

17. The method of claim 1, wherein the disease is vaginal dysplasia.

18. The method of claim 1, wherein the disease is vaginal carcinoma.

19. The method of claim 1, wherein the disease is vulvar dysplasia.

20. The method of claim 1, wherein the disease is vulvar carcinoma.

21. The method of claim 1, wherein the disease is cervical dysplasia.

22. The method of claim 1, wherein the disease is Kaposi's sarcoma.

23. The method of claim 1, wherein the compound of Formula I is administered as a pharmaceutical composition comprising the compound of Formula I at a concentration by weight within a range from about 0.01% to about 20% or or at a patient weight dosage within a range from about 1 mg/kg to about 100 mg/kg, together with a pharmaceutically acceptable carrier.

24. The method of claim 23, wherein the compound of Formula I is administered as a pharmaceutical composition comprising the compound of Formula I at a concentration by weight within a range from about 1% to about 10% or or at a patient weight dosage within a range from about 1 mg/kg to about 60 mg/kg, together with a pharmaceutically acceptable carrier.

25. The method of claim 24, wherein the pharmaceutical composition is an oral or parenteral pharmaceutical composition.

26. The method of claim 24, wherein the pharmaceutical composition is a topical pharmaceutical composition.

27. The method of claim 26, wherein the topical pharmaceutical composition further comprises petroleum jelly or dimethyl sulfoxide.

28. The method of claim 24, wherein the topical pharmaceutical composition further comprises at least one compound selected from the group consisting of cell differentiating agents, anti-proliferative agents, mitochondrial inhibitors, topical steroids, immunosuppressive compounds, JAK2 inhibitors, JAK3 inhibitors, parathyroid hormone-related protein agonists, cell adhesion blockers, derivatives thereof, and combinations thereof.

29. The method of claim 24, wherein the topical pharmaceutical composition further comprises a cell differentiating agent selected from at least one of retinoic acid, retinoic acid derivative, vitamin D, or vitamin D analog.
